(19) United States
(12) Patent Application Publication
(10) Pub. No.: US 2025/0177132 A1
(43) Pub. Date: Jun. 5, 2025

(54) DELIVERY APPARATUS FOR A MECHANICALLY EXPANDABLE PROSTHETIC HEART VALVE

(71) Applicant: EDWARDS LIFESCIENCES CORPORATION, Irvine, CA (US)

(72) Inventor: Eitan Atias, Netanya (IL)

(73) Assignee: EDWARDS LIFESCIENCES CORPORATION, Irvine, CA (US)

(21) Appl. No.: 19/048,713

(22) Filed: Feb. 7, 2025

Related U.S. Application Data
(63) Continuation of application No. PCT/US2023/030404, filed on Aug. 16, 2023.
(60) Provisional application No. 63/399,555, filed on Aug. 19, 2022.

(51) Int. Cl. A61F 2/24

(52) U.S. Cl. CPC A61F 2/243; A61F 2/2418

(57) ABSTRACT

A delivery apparatus for implanting a prosthetic valve is disclosed. The delivery apparatus comprises at least one actuation assembly configured to actuate an actuator member of a prosthetic valve, the actuator member configured to be rotated via rotation of a head thereof to radially expand or compress a frame the prosthetic valve, the actuation assembly comprising: an outer sleeve member having a lumen, wherein a distal end portion of the sleeve member comprises internal threads; a driver member coaxially disposed within the lumen of the sleeve member, the driver member being rotatable relative to the sleeve member, wherein a distal end portion of the driver member comprises a socket configured to receive the head of the actuator member, wherein the actuation assembly is configured to be transitioned between an engaged position and a disengaged position with the frame.

### CROSS-REFERENCE TO RELATED APPLICATIONS

This application is a continuation of PCT Application No. PCT/US2023/030404, filed Aug. 16, 2023, which claims the benefit of U.S. Provisional Application No. 63/399,555, filed Aug. 19, 2022, both of which are incorporated by reference herein.

### FIELD

The present disclosure relates to apparatus and methods for delivering, expanding, implanting, and deploying implantable, radially expandable prosthetic devices, such as mechanically expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (for example, for repositioning and/or retrieval). However, some known devices and methods can cause rotation or movement of the prosthetic valve during expansion.

Despite the recent advancements in percutaneous valve technology, there remains a need for improved transcatheter heart valves and delivery devices for such valves.

### SUMMARY

Described herein are prosthetic heart valves, delivery apparatuses, and methods for implanting prosthetic heart valves. The disclosed delivery apparatus and methods can, for example, reduce the complexity and/or the time needed to implant a prosthetic heart valve. The disclosed delivery apparatuses are relatively simple and easy to use and include various safeguards, which can help to ensure that the prosthetic heart valve is safely and securely implanted. As such, the devices and methods disclosed herein can, among other things, overcome one or more of the deficiencies of typical delivery apparatuses for mechanically expandable prosthetic valves.

A delivery apparatus for a prosthetic implant can comprise a handle and one or more shafts coupled to the handle.

In some examples, a delivery apparatus can comprise an actuation assembly configured to actuate an actuator member of a prosthetic valve, wherein the actuation assembly comprises a sleeve member and a driver member.

In some examples, the sleeve member comprises internal threads for engaging external threads of an actuator member of a prosthetic valve.

In some examples, the driver member comprises a socket for engaging a head of an actuator member of a prosthetic valve.

In some examples, the driver member is configured to rotate independent of and relative to the sleeve member.

In some examples, the driver member is coaxially disposed within the sleeve member.

A prosthetic valve can comprise a radially expandable and compressible frame, a valvular structure disposed within the frame, and at least one rotatable actuator for expanding and compressing the frame.

In some examples, the prosthetic valve includes a coupling member that is coupled to the actuator.

In some examples, the coupling member has external threads for the sleeve member comprises internal threads for engaging internal threads of a sleeve member of an actuation assembly.

In some examples, the actuator is rotatable relative to the frame in a first rotational direction to produce radial expansion of the frame from the radially compressed state to the radially expanded state, and the coupling member is fixed relative to the frame in the first rotational direction.

In some examples, a delivery apparatus for implanting a prosthetic valve is disclosed. The delivery apparatus comprises: at least one actuation assembly configured to actuate an actuator member of a prosthetic valve, the actuator member configured to be rotated via rotation of a head thereof to radially expand or compress a frame the prosthetic valve, the actuation assembly comprising: a sleeve member having a lumen, wherein a distal end portion of the sleeve member comprises internal threads; and a driver member coaxially disposed within the lumen of the sleeve member, the driver member being rotatable relative to the sleeve member, wherein a distal end portion of the driver member comprises a socket configured to receive the head of the actuator member, wherein the actuation assembly is configured to be transitioned between an engaged position and a disengaged position with the frame.

In some examples, a delivery apparatus for a prosthetic valve comprises: a handle; a driver having a proximal end portion coupled to the handle and a distal end portion comprising an engagement head to releasably couple the driver to an actuator of the prosthetic valve; and a sleeve member operatively coupled to the driver, the sleeve member having a proximal end portion coupled to the handle and a distal end portion comprising internal threads to releasably coupled the sleeve member to a frame of the prosthetic valve, wherein the driver extends through the sleeve member.

In some examples, a prosthetic valve comprises: a radially expandable and compressible frame comprising a plurality of interconnected struts, wherein the frame is radially expandable and compressible between a radially compressed state and a radially expanded state; a valvular structure disposed within the frame and configured to regulate the flow of blood through the frame in one direction; at least one rotatable actuator operatively coupled to the frame, wherein the actuator is rotatable relative to the frame in a first rotational direction to produce radial expansion of the frame from the radially compressed state to the radially expanded state; and at least one coupling member having external threads, wherein the coupling member is coupled to the actuator and the frame, wherein the coupling member is fixed relative to the frame in the first rotational direction.

In some examples, an assembly comprises: a prosthetic valve having a radially expandable and compressible frame, a valvular structure disposed within the frame and configured to regulate the flow of blood through the frame in one direction at least one actuator operatively coupled to the frame, and a threaded coupling member, wherein the actuator is configured to be rotated, via rotation of a head thereof, to radially expand or compress the frame; and a delivery device comprising a sleeve member having internal threads and a driver member at least partially disposed within the sleeve member, wherein the sleeve member is configured to form a releasable threaded connection with the coupling member and the driver member is configured to releasably engage the actuator and rotate the actuator upon rotation of the driver member.

In some examples, a method of implanting a prosthetic valve comprises: engaging internal threads of a sleeve coupled to a handle with external threads of a frame of the prosthetic valve; engaging a socket of a driver coupled to the handle with a head of an actuator of the frame; delivering the prosthetic valve to an implantation location within a patient's body; radially expanding the prosthetic valve to a functional size; and releasing the sleeve from the frame and releasing the driver from the actuator.

In some examples, a method of implanting a prosthetic valve comprises: engaging an end portion of a sleeve coupled to a handle with an end portion of a frame of the prosthetic valve; preventing rotation of an output gear of a gear train disposed within the handle and operatively coupled to the sleeve; delivering the prosthetic valve to an implantation location within a patient's body; radially expanding the prosthetic valve to a functional size; then allowing rotation of the output gear; and releasing the end portion of the sleeve from the end portion of the frame.

In some examples, an assembly comprises: a prosthetic heart valve comprising a frame and at least one actuator coupled to the frame, the frame comprising a threaded engagement portion having external threads, the actuator operable to move the frame between a radially expanded configuration and a radially compressed configuration; at least one actuation assembly comprising: an actuator driver configured to releasably engage the at least one actuator; and a sleeve member operatively coupled to the actuator driver and movable between a first position in which the actuator driver is retained in engagement with the at least one actuator and a second position in which the actuator driver is released from engagement from the at least one actuator, the sleeve member comprising internal threads configured for threaded engagement with the external threads of the frame in the first position.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of a prosthetic device, according to one example.

FIG. 1B is a side elevation view of a frame of the prosthetic device of FIG. 1A, according to one example.

FIGS. 2A-2B are perspective views of a proximal end portion of the frame of the prosthetic device of FIGS. 1A-1B, according to one example.

FIG. 3 is a side elevation view of a delivery apparatus for a prosthetic device, according to one example.

FIGS. 4A and 4B are perspective views of a portion of the prosthetic device of FIGS. 1A-1B and actuation assemblies configured for actuation thereof, according to one example.

FIG. 5A is a cross-sectional view illustrating engagement between a portion of the prosthetic device of FIGS. 1A-1B and the actuation assembly of FIGS. 4A-4B.

FIG. 5B is a perspective view of a distal end of the actuation assembly of FIGS. 4A-4B.

FIG. 6 is a perspective view of a delivery apparatus for a prosthetic device, according to one example.

FIGS. 7A-7B are top elevation views of the delivery apparatus of FIG. 6.

FIG. 8 is a side elevation view of the delivery apparatus of FIG. 6.

FIG. 9 is a cross-sectional view of the delivery apparatus of FIG. 6 illustrating an example gear train.

FIGS. 10A and 10B are cross-sectional views of the delivery apparatus of FIG. 6 illustrating an example gear train.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic devices disclosed herein can be advanced through a patient's vasculature on a delivery apparatus. The prosthetic devices can include one or more expansion and locking mechanisms that can be actuated using the delivery apparatus to radially expand the prosthetic device and lock the prosthetic devices in one or more radially expanded states. As one example, the prosthetic devices can be crimped on or retained by the delivery apparatus in a radially compressed state during delivery, and then radially expanded (and axially shortened) to a radially expanded state once the prosthetic devices reach the implantation site. It is understood that the prosthetic devices disclosed herein may be used with a variety of implant delivery apparatus.

In some examples, the delivery apparatus disclosed herein includes one or more multi-layer delivery and actuation mechanisms. For example, a delivery apparatus can include an actuation assembly having two concentric or coaxial components, including an outer sleeve member and an inner driver. The inner driver can be coaxial with and disposed within a lumen of the outer sleeve member. The inner driver can be rotatable relative to the outer sleeve member. A proximal end of each of the sleeve shaft and the driver shaft can be coupled to a handle or other controls of the delivery apparatus to enable the surgeon to steer the actuation assembly during transcatheter implant delivery and to control the radial expansion and/or radial compression of a frame of a mechanically expandable prosthetic valve (for example, control rotation of the inner driver and the outer sleeve member, etc.).

During use of the actuation assembly, the sleeve member can be utilized as a stationary coupler configured to releasably engage (for example, via a threaded engagement) a portion of the frame of the mechanically expandable prosthetic valve. The inner driver can be utilized as a rotatable coupler configured to releasably engage a respective portion of a head of an actuator (for example, a threaded member) of the frame and cause radial expansion and/or radial compression of the frame. The actuation assembly can be configured to limit unwanted axial and rotational movement of the prosthetic heart valve during radial expansion and/or compression of the frame, as well as during separation of the actuation assembly from the prosthetic valve after radial expansion of the frame. Exemplary delivery apparatus and actuation assemblies and associated methods of use will be discussed in more detail below.

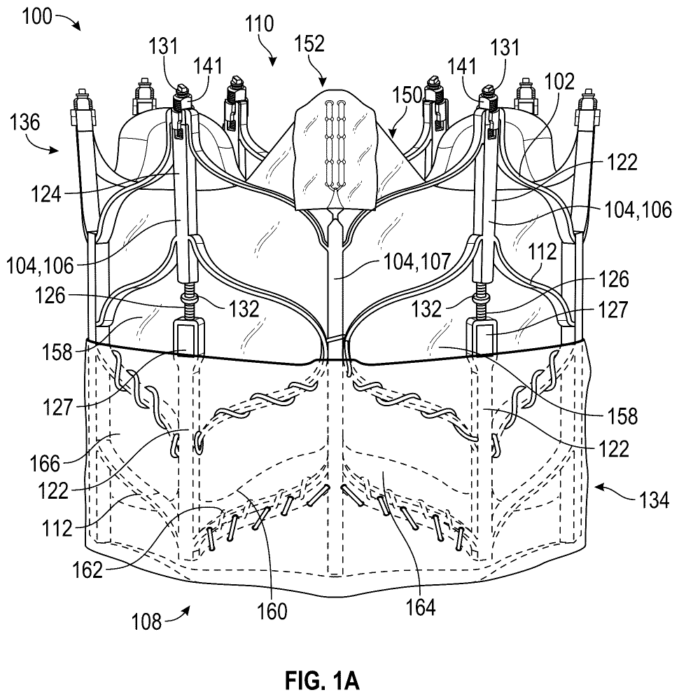

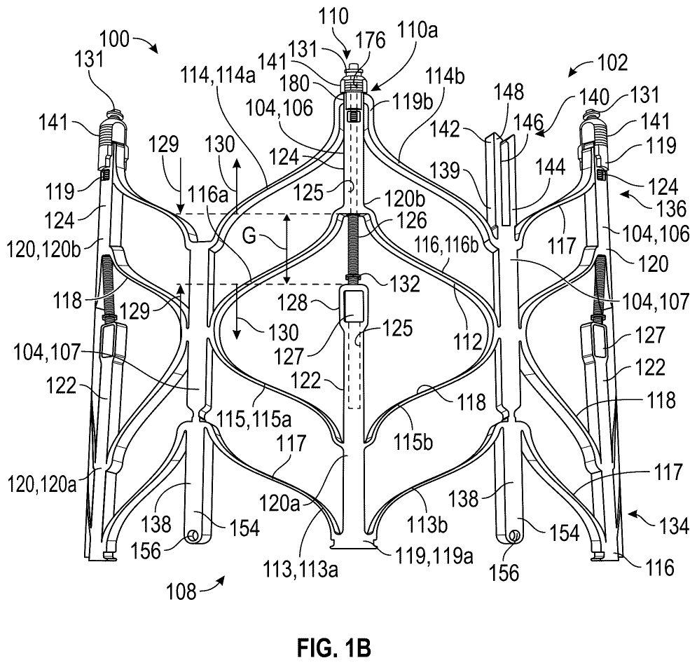

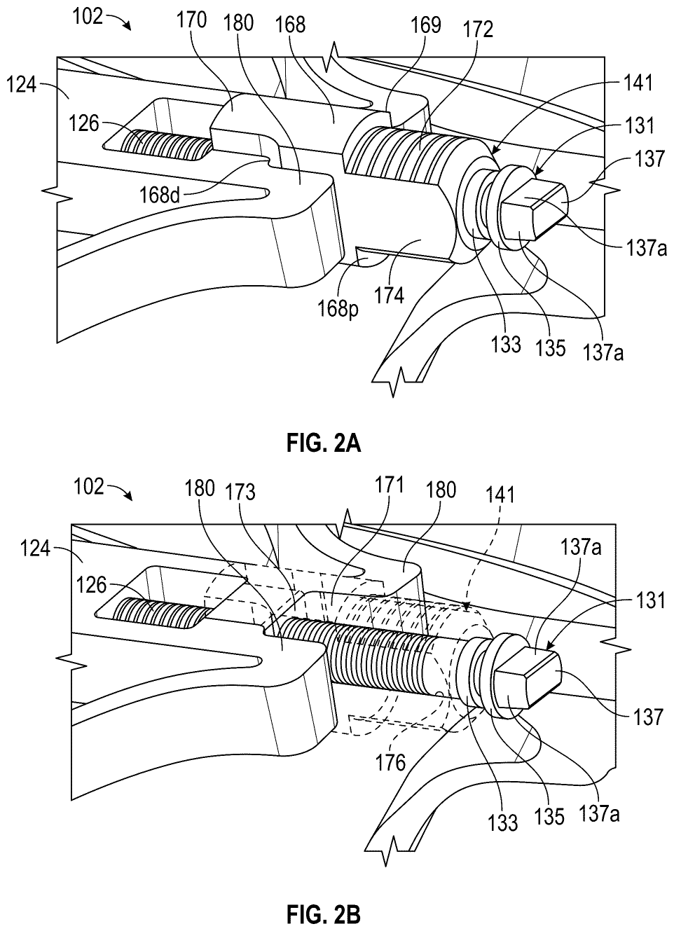

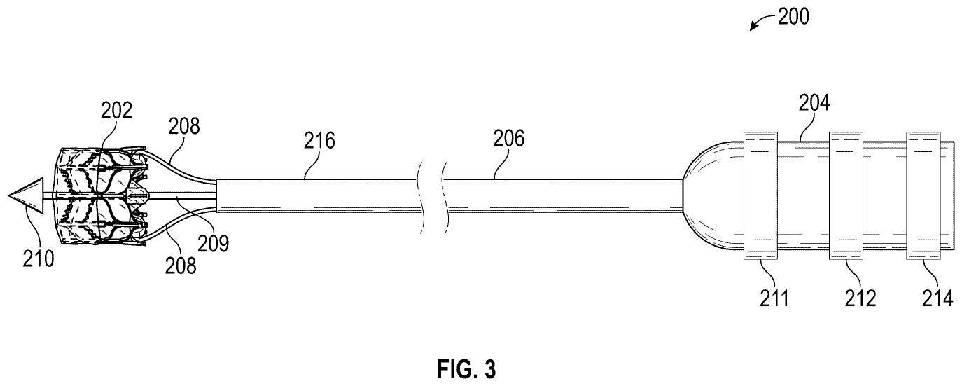

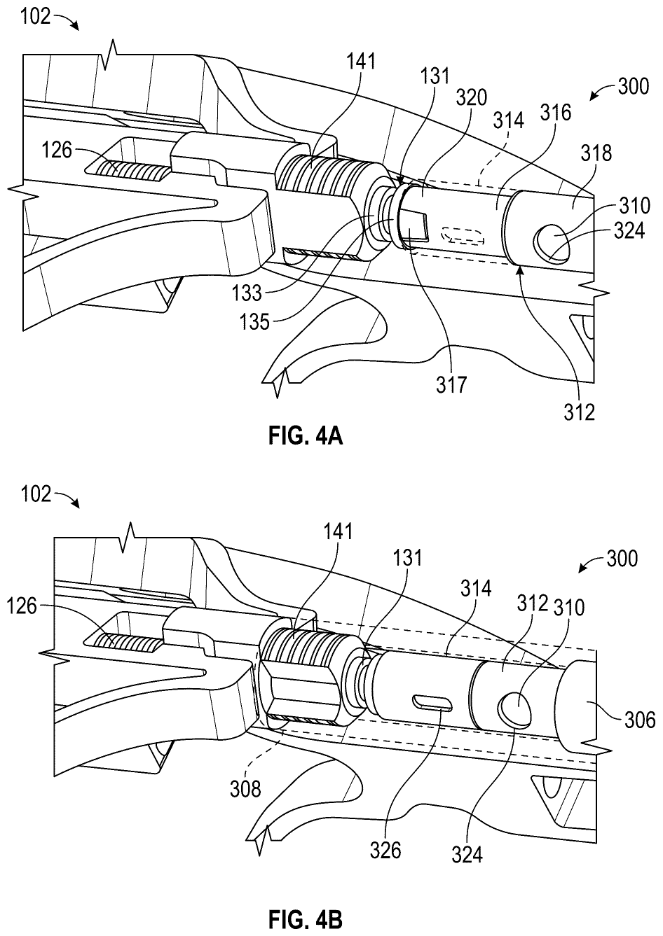

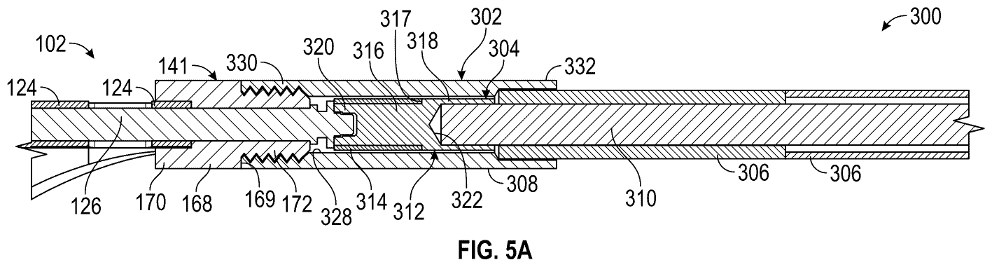

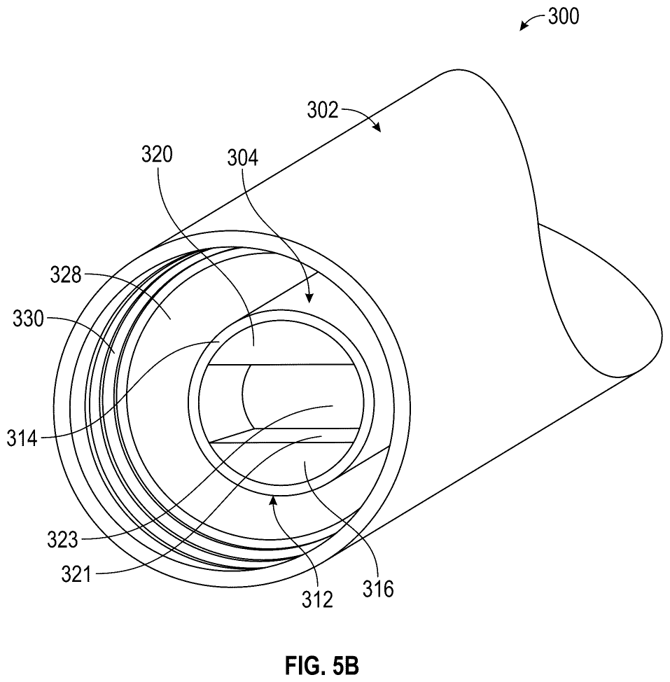

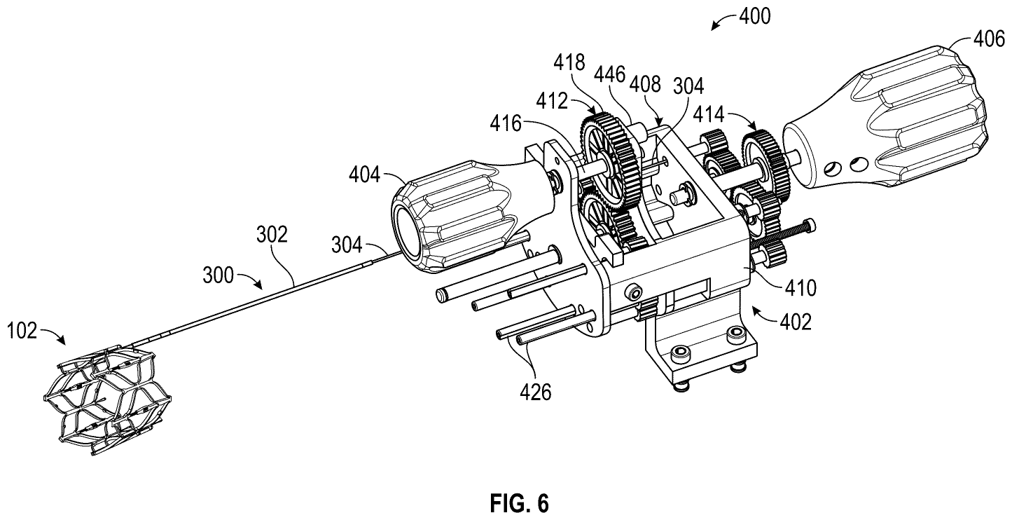

FIGS. 1A-1B illustrate an exemplary prosthetic device (for example, prosthetic heart valve) that can be advanced through a patient's vasculature, such as to a native heart valve, by a delivery apparatus, such as the exemplary delivery apparatus shown in FIG. 2. The frame of the prosthetic device can include mechanical expansion and locking mechanisms that can be integrated into the frame-specifically, into axially extending posts of the frame. The mechanical expansion and/or locking mechanisms can be removably coupled to, and/or actuated by, the delivery apparatus. Specifically, the mechanical expansion and/or locking mechanisms can be removably coupled to an actuation assembly (such as the actuation assembly 300 illustrated in FIGS. 4A-5B) of the delivery apparatus, which in turn can be actuated by a physician by adjusting and/or manipulating one or more input devices (for example, one or more knobs, buttons, drawstrings, etc.) that can be included on a handle (such as the handle 204 illustrated in FIG. 3, the handle 402 illustrated in FIGS. 6-8C, etc.) of the delivery apparatus.

### Examples of the Disclosed Technology

FIGS. 1A and 1B illustrate an example of a prosthetic device 100 (which also may be referred to herein as “prosthetic heart valve 100” and/or “prosthetic valve 100”). Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic valve 100 comprises a frame 102. FIG. 1A shows the frame 102 with a valvular structure (for example, leaflets) and an optional skirt assembly, while FIG. 1B shows the frame 102 with these components removed. While only one side of the frame 102 is depicted in FIG. 1B, it should be appreciated that the frame 102 forms an annular structure having an opposite side that is substantially identical to the portion shown in FIG. 1B.

The frame 102 comprises a plurality of axially extending posts 104, one or more of which can be configured as integral expansion and locking mechanisms 106 (also referred to herein as “actuators 106”) and/or one or more of which can be configured as support posts 107. Specifically, the actuators 106 (which can be used to radially expand and/or radially compress the prosthetic device 100) can be integrated into the frame 102 of the prosthetic device 100, thereby reducing the crimp profile and/or bulk of the prosthetic device 100 (making the prosthetic device 100 have an overall lower profile). Integrating the expansion and locking mechanisms 106 into the frame 102 can also simplify the design of the prosthetic device 100, making the prosthetic device 100 less expensive and/or easier to manufacture.

The frame 102 has a distal end 108 and a proximal end 110. In some examples, as shown in FIGS. 1A and 1B, the distal end 108 can be an inflow end and the proximal end can be an outflow end, such as when the prosthetic device 100 is configured to replace a native aortic valve and the prosthetic device 100 is delivered to the native aortic valve via a retrograde, transfemoral delivery approach (for example, through a femoral artery and the aorta). However, in some examples, the distal end 108 can be the outflow end and the proximal end 110 can be the inflow end, such as when the prosthetic device 100 is delivered to the native aortic valve via a transapical delivery approach, or when the prosthetic valve is configured to replace a native mitral valve and is delivered to the native mitral valve in a trans-septal delivery approach in which the delivery apparatus and the prosthetic valve are advanced into the right atrium, through the atrial septum, and into the left atrium, wherein the right atrium may be accessed via a femoral vein and inferior vena cava or via the superior vena cava.

The posts 104 can be coupled together by a plurality of circumferentially extending link members or struts 112. Each strut 112 extends circumferentially between adjacent posts 104 to connect all of the axially extending posts 104. As one example, the prosthetic device 100 can include equal numbers of support posts 107 and expansion and locking mechanisms 106 and the expansion and locking mechanisms 106 and the support posts 107 can be arranged in an alternating order such that each strut 112 is positioned between one of the expansion and locking mechanisms 106 and one of the support posts 107 (that is, each struts 112 can be coupled on one end to one of the posts 104 that is configured as one of the expansion and locking mechanisms 106 and can be coupled on the other end to one of the support posts 107). However, the prosthetic device 100 can include different numbers of support posts 107 and expansion and locking mechanisms 106 and/or the expansion and locking mechanisms 106 and the support posts 107 can be arranged in a non-alternating order, in some examples.

As illustrated in FIG. 1B, the struts 112 can include a first row of struts 113 at or near the distal end 108 of the prosthetic device 100, a second row of struts 114 at or near the proximal end 110 of the prosthetic device 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the posts 104. Each second cell 118 can be formed by two struts 115a, 115b of the third row of struts 115 and two struts 116a, 116b of the fourth row of struts 116. As illustrated in FIG. 1B, each second cell 118 can be disposed within one of the first cells 117 (that is, the struts 115a-116b forming the second cells 118 are disposed between the struts forming the first cells 117 (that is, struts 113a, 113b and the struts 114a, 114b), closer to an axial midline of the frame 102 than the struts 113a-114b).

As illustrated in FIG. 1B, the struts 112 of frame 102 can comprise a curved shape. Each of the first cells 117 can have an axially-extending elliptical shape including first and second apices 119 (for example, proximal apex 119a and distal apex 119b) disposed at the major vertices of the ellipse. In the illustrated example, the distal apex 119 of each of the first cells 117 can include two extensions or arms 180 (also referred to herein as proximal end portions) that extend proximally from the proximal end 110 of the frame 102. As will be described in more detail below, the pair of arms 180 can extend proximally along a length of a coupling member 141 disposed on the threaded rod 126, such that the coupling member 141 and the threaded rod 126 are at least partially disposed between the pair of extensions 180 and at least partially recessed relative to the proximal end 110 of the frame 102. Exemplary prosthetic valve frames including proximal posts having a recess (for example, a recess at the proximal end of the post defined by a pair of arms or extensions) that is configured to receive or house a portion of a threaded rod (or coupling member) and that can be used in combination with the exemplary prosthetic devices and actuator and/or delivery assemblies disclosed herein are described in U.S. Provisional Patent Application No. 63/277,959, which is incorporated by reference herein.

Each second cell 118 can have a circumferentially-extending elliptical shape including first and second apices 120 (for example, proximal apex 120a and distal apex 120b) disposed at the minor vertices of the ellipse. In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

Some of the posts 104, such as the posts that are configured as the integral expansion and locking mechanisms 106, can be discontinuous and can each include a first (distal) strut or post 122 and a second (proximal) strut or post 124 that are axially separated from one another by a gap G (FIG. 1B). The first post 122 (that is, the lower post shown in FIG. 1B) can extend axially from the distal end 108 of the prosthetic device 100 toward the second post 124, and the second post 124 (that is, the upper post shown in FIG. 1B) can extend axially from the proximal end 110 of the frame 102 toward the first post 122. Each first or distal post 122 can be axially aligned with a corresponding second or proximal post 124 and one or both of the posts 122, 124 can include an inner bore 125, so that, for example, the axially aligned pair of posts 122, 124 can receive an actuator member, such as in the form of a substantially straight threaded rod 126 as shown in the illustrated example. The threaded rod 126 also may be referred to herein as an actuator, an actuator member, and/or a screw actuator.

The threaded rod 126 can be coupled to the distal post 122 and/or the proximal post 124. In some examples, the rod 126 can be rotatably coupled to the distal post 122 such that the rod 126 can only be rotated relative to the distal post 122 to move the rod 126 axially relative to the distal post but otherwise cannot move relative to the distal post 122 (for example, the threaded rod 126 cannot slide axially relative to the distal post 122 or move circumferentially and/or radially relative to the distal post 122). Further, the rod 126 can be restrained radially and/or circumferentially by the proximal post 124 such that the rod 126 can only be rotated and/or slid axially relative to the proximal post 124 but otherwise cannot move relative to the proximal member 124 (for example, radially and/or circumferentially). In some examples, the threaded rod 126 can be inserted through the inner bore 125 of one of the proximal posts 124 and into a stationary nut 127 and/or bore 125 included in an axially aligned one of the distal posts 122. However, in other examples, the threaded rod 126 need not extend through a bore in the proximal post 124 and instead can be restrained and/or restrained by the proximal post 124 using other suitable structures such as guides, straps, loops, collars, etc.

The stationary nut 127 can be included at a proximal end portion 128 of the distal post 122. In some examples, the threaded rod 126 can extend distally past the nut 127 into the inner bore 125 of the distal post 122. The stationary nut 127 can be held in a fixed position relative to the distal post 122 such that the nut 127 does not rotate relative to the distal post 122. In this way, whenever the threaded rod 126 is rotated (for example, by a physician), the threaded rod 126 can rotate relative to both the stationary nut 127 and the distal post 122. The nut 127 can include a threaded bore that that is configured to engage the threads of the threaded rod 126 to prevent the rod 126 from moving axially relative to the nut 127 and the post 122 unless the threaded rod 126 is rotated relative to the nut 127. Thus, the threaded rod 126 can be retained/held by the nut 127 and can only be moved relative to the nut 127 and/or the post 122 by rotating the threaded rod 126 relative to the nut 127 and/or the post 122. In some examples, in lieu of using the nut 127, at least a portion of the inner bore 125 of the distal post 122 can be threaded. For example, the proximal end portion 128 of the distal post 122 can comprise inner threads configured to engage the threaded rod 126 such that rotation of the threaded rod causes the rod 126 to move axially relative to the first post 122.

When the threaded rod 126 is inserted into and/or otherwise coupled to a pair of axially aligned posts 122, 124, the pair of axially aligned posts can serve as one of the expansion and locking mechanisms 106. In some examples, one of the threaded rods 126 can be inserted through each pair of axially aligned posts 122, 124 so that all of the posts 122, 124 serve as expansion and locking mechanisms 106. As just one example, the prosthetic device 100 can include six pairs of posts 122, 124, and each of the six pairs of posts 122, 124 can be configured as one of the expansion and locking mechanisms 106 for a total of six expansion and locking mechanisms 106. In some examples, not all pairs of posts 122, 124 need be expansion and locking mechanisms (that is, actuators). If a pair of posts 122, 124 is not used as an actuator, a threaded rod 126 need not extend through the posts 122, 124 of that pair. In the description that follows, the first and second (or distal and proximal) posts 122, 124, respectively, that are used as actuators (that is, those that include threaded rods 126), can be referred to as first and second (or distal and proximal) actuator members 122, 124, or more simply, first and second (or distal and proximal) members 122, 124.

The threaded rod 126 can be rotated relative to the nut 127 and/or the first post 122 to axially foreshorten and/or axially elongate the frame 102, thereby radially expanding and/or radially compressing, respectively, the prosthetic device 100. Specifically, when the threaded rod 126 is rotated relative to the nut 127 and/or the first post 122, the first and second members 122, 124 can move axially relative to one another, thereby widening or narrowing the gap G (FIG. 1B) separating the posts 122, 124, and thereby radially compressing or radially expanding the prosthetic device 100, respectively. Thus, the gap G (FIG. 1B) between the first and second posts 122, 124 narrows as the frame 102 is radially expanded and widens as the frame 102 is radially compressed. In other words, rotation of the threaded rod 126 in a first direction (for example, clockwise) can cause corresponding axial movement of the first and second members 122, 124 toward one another (as shown by arrows 129 in FIG. 1B), thereby radially expanding the frame 102, while rotation of the threaded rod 126 in an opposite second direction (for example, counterclockwise) causes corresponding axial movement of the first and second members 122, 124 away from one another (as shown by arrows 130 in FIG. 1B), thereby radially compressing the frame.

The threaded rod 126 can extend proximally past the proximal end of the proximal post 124 and through an inner bore 176 of a coupling member 141 disposed on the proximal end of the proximal post 124. The coupling member 141 can be integrally formed on or fixedly coupled to the proximal post 124 such that the coupling member 141 does not move relative to the proximal post 124. Additional details of the coupling member 141 will be described below with reference to FIGS. 2A-2B.

Further, the threaded rod 126 can include a head portion 131 at its proximal end. The head portion 131 of the threaded rod 126 can be proximal to the coupling member 141. The head portion 131 of the threaded rod 126 can serve at least two functions. First, as will be described in greater detail below with reference to FIGS. 3A-5B, the head portion 131 can releasably couple the threaded rod 126 to a respective actuation assembly of the delivery apparatus that can be used to drive rotation of the head portion 131 and the threaded rod 126 to radially expand and/or radially compress the prosthetic device 100. Second, the head portion 131 can prevent the coupling member 141 and the proximal post 124 from moving proximally relative to the threaded rod 126 and can apply a distally directed force to the coupling member 141 and the proximal post 124, such as when radially expanding the prosthetic device 100. Specifically, the head portion 131 can have a width greater than a diameter of an inner bore 176 of the coupling member 141 such that the head portion 131 is prevented from moving into the inner bore 176 of the coupling member 141 and/or the inner bore 125 of the proximal post 124. Thus, as the threaded rod 126 is threaded farther into the nut 127, the head portion 131 of the threaded rod 126 draws closer to the nut 127 and the first post 122, thereby drawing the proximal post 124 towards the distal post 122, and thereby axially foreshortening and radially expanding the prosthetic device 100.

The threaded rod 126 also can include a stopper 132 (for example, in the form of a nut or flange) disposed thereon. The stopper 132 can be disposed on the threaded rod 126 such that the stopper 132 sits within the gap G. Further, the stopper 132 can be integrally formed on or fixedly coupled to the threaded rod 126 such that the stopper 132 does not move relative to the threaded rod 126. Thus, the stopper 132 can remain in a fixed position on the threaded rod 126 such that the stopper 132 moves in lockstep with the threaded rod 126. The stopper 132 can apply a proximally directed force to the proximal post 124 to radially compress the prosthetic device 100. Specifically, during crimping/radial compression of the prosthetic valve 100, the threaded rod 126 can be rotated in the second direction (for example, counterclockwise) causing the stopper 132 to push against (that is, provide a proximally directed force to) the distal end of the proximal post 124, thereby causing the proximal post 124 to move away from the distal post 122, and thereby axially elongating and radially compressing the prosthetic device 100.

Thus, the proximal posts 124 and coupling members 141 can be axially retained and/or restrained between the head portion 131 and the stopper 132. That is, each proximal post 124 and coupling member 141 pair can be (alternatively) restrained at its proximal end by the head portion 131 and at its distal end by the stopper 132. In this way, the head portion 131 can apply a distally directed force to the coupling member 141 (which is transferred to the proximal post 124) to radially expand the prosthetic device 100 when the head portion 131 is rotated in the first direction (for example, clockwise), while the stopper 132 can apply a proximally directed force to the proximal post 124 to radially compress the prosthetic device 100 when the head portion 131 is rotated in the second direction (for example, counterclockwise). As explained above, radially expanding the prosthetic device 100 axially foreshortens the prosthetic device 100, causing a distal end portion 134 and a proximal end portion 136 of the prosthetic device 100 to move towards one another axially, while radially compressing the prosthetic device 100 axially elongates the prosthetic device 100, causing the distal and proximal end portions 134, 136 to move away from one another axially.

As also introduced above, some of the posts 104 can be configured as support posts 107 (also referred to as “axial struts”). As shown in FIG. 1B, the support posts 107 can extend axially between the distal and proximal end portions 134, 136 of the frame 102 and can have a distal end portion 138 and a proximal end portion 139. The proximal end portion 139 of one or more support posts 107 can include a commissure support structure or member 140. The commissure support member 140 can comprise first and second commissure arms 142, 144 defining a commissure opening 146 between them. The proximal end of each commissure arm 142, 144 can include a tooth 148 extending into the commissure opening 146. The commissure opening 146 can extend radially through a thickness of the post 107 and can be configured to accept a portion of a valvular structure 150 (for example, a commissure 152) to couple the valvular structure 150 to the frame 102. For example, each commissure 152 can be mounted to a respective commissure support member 140, such as by inserting a pair of commissure tabs of adjacent leaflets through the opening 146 and suturing the commissure tabs to each other and/or the arms 142, 144. In some examples, the opening 146 can be fully enclosed by the post 107 (for example, not extending to the proximal edge) such that a portion of the valvular structure 150 can be slid radially (rather than axially) into the commissure opening 146. The teeth 148 can help retain the commissure 152 within the commissure opening 146. In the illustrated example, the commissure opening 146 has a substantially rectangular shape and extends to the distal end of the post 104. However, in some examples, the commissure opening can have any of various shapes (for example, square, oval, square-oval, triangular, L-shaped, T-shaped, C-shaped, etc.).

Though only one support post 107 comprising a commissure support member 140 is shown in FIG. 1B, it should be noted that the frame 102 can comprise any number of support posts 107, any number of which can be configured as commissure support members 140. For example, the frame 102 can comprise six support posts 107, three of which are configured as commissure support members 140. However, in some examples, the frame 102 can comprise more or less than six support posts 107 and/or more or less than three commissure support members 140.

The opposite distal end portion 138 of each support post 107 can comprise an extension 154 that extends toward the distal end of the frame 102. The extension 154 can comprise an aperture or opening 156 extending radially through a thickness of the extension 154. In some examples, the extension 154 can extend such that a distal edge of the extension 154 aligns with or substantially aligns with the distal end of the frame 102. In use, the extension 154 can prevent or mitigate portions of an outer skirt from extending radially inwardly and thereby prevent or mitigate any obstruction of flow through the distal end portion 134 of the frame 102 caused by the outer skirt. The extensions 154 can further serve as supports to which portions of the inner and/or outer skirts can be coupled. For example, sutures used to connect the inner and/or outer skirts can be wrapped around the extensions 154 and/or can extend through apertures 156.

As illustrated in FIG. 1A, the prosthetic valve 100 can further include the valvular structure 150, which is coupled to and supported inside the frame 102. The valvular structure 150 is configured to regulate the flow of blood through the prosthetic valve 100, from the inflow end to the outflow end. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form the commissures 152, each of which can be secured to a respective post 104 (for example, to a support post 107) and/or to other portions of the frame 102.

In the example depicted in FIG. 1A, the valvular structure 150 includes three leaflets 158, which can be arranged to collapse in a tricuspid arrangement. Each leaflet 158 can have a proximal edge portion 160. As shown in FIG. 1A, the proximal edge portions 160 of the leaflets 158 can define an undulating, curved scallop edge that follows or tracks portions of the struts 112 of frame 102 in a circumferential direction when the frame 102 is in the radially expanded configuration. The proximal edges 160 of the leaflets can be referred to as a “scallop line.”

As shown in FIG. 1A, the proximal edge portions 160 of the leaflets 158 can be sutured to an inner skirt 164 generally along the scallop line. The inner skirt 164 can in turn be sutured, via one or more sutures 162, for example, to adjacent struts 112 of the frame 102. In some examples, the leaflets 158 can be sutured directly to the frame 102 along the scallop line.

The prosthetic valve 100 can further include one or more skirts or sealing members. For example, the prosthetic valve 100 can include the inner skirt 164, mounted on the radially inner surface of the frame 102. The inner skirt 164 can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic device 100. The prosthetic device 100 can further include an outer skirt 166 mounted on the outer surface of the frame 102. The outer skirt 166 can function as a sealing member for the prosthetic device 100 by scaling against the tissue of the native valve annulus and helping to reduce paravalvular leakage past the prosthetic device 100. The inner and outer skirts 164, 166 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valves can be found, for example, in U.S. Patent Application No. 62/854,702 and PCT Patent Application No. US2020/024559, each of which is incorporated by reference herein.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

As introduced above, coupling members 141 can be disposed on the proximal ends of the proximal posts 124. FIGS. 2A-2B illustrate an example of a coupling member 141 that is coupled to a proximal post 124. In the illustrated example, the coupling member 141 can be fixedly coupled to the proximal post 124, such that it does not move relative to the proximal post 124 (for example, in a rotational direction, etc.). In some examples, the coupling member 141 can be integrally formed with the proximal post 124 (for example, such that the proximal post 124 includes external threads at a proximal end thereof, etc.). The coupling member 141 is shown transparently in FIG. 2B for purposes of illustration.

As can be seen in FIGS. 2A and 2B, in the present example, the coupling member 141 can comprise a base 168, extensions 170 (or arms) extending distally from a distal end 168d of the base 168, and a threaded engagement portion 172 having external threads extending proximally from a proximal end 168p of the base 168. The base 168 can have a relatively larger outer diameter than the threaded engagement portion 172. As such, an annular shoulder 169 can be formed between the base 168 and the threaded engagement portion 172 (for example, at the proximal end 168p of the base 168). In this manner, the coupling member 141 can have an overall stepped configuration. In some examples, the threaded engagement portion 172 and the base 168 can have the same outer diameter. In some examples, the threaded engagement portion 172 can have a relatively larger outer diameter than the base 168. In some instances, the base 168 can also comprise the external threads (for example, such that the coupling member 141 includes extensions 170 extending distally from a member that is threaded along its entire length, etc.).

The coupling member 141 can include one or more facets or flat portions 174 that are evenly and circumferentially spaced apart at the exterior surface thereof. For example, as depicted, the coupling member 141 can include two facets 174, each positioned at 180° relative to other facet 174, on the exterior surface of the base 168 and the threaded engagement portion 172. Further, as depicted, the coupling member 141 can include two extensions 170 that can be evenly and circumferentially spaced apart and can be positioned at 90° relative to the facets 174.

The coupling member 141 can be disposed between the arms 180 of the proximal post 124. For example, the arms 180 can define a socket or recess at the proximal end of the post 124. As best shown in FIG. 2A, the base 168 of the coupling member 141 can be received within the socket of the proximal post 124, such that the base 168 is recessed relative to the proximal end 110 of the frame 102. In particular, the facets 174 can be aligned with or abut inner surfaces 171 of the arms 180 and a surface at the distal end 168d of the base 168 can be aligned with or abut a proximal face 173 of the proximal post 124 (FIG. 2B). The coupling member 141 can be retained on the post 124, such as via a press-fit between the base 168 and the socket and/or welding the coupling member to the post.

The coupling member 141 can include an inner bore 176 that is axially aligned with the inner bore 125 of the proximal post 124. The threaded rod 126 can be coupled to the coupling member 141. Specifically, the threaded rod 126 can extend through the inner bore 176 of the coupling member 141 as well as the inner bore 125 of the proximal post 124. In some examples, the rod 126 can be restrained radially and/or circumferentially by the inner bore 176 of the coupling member 141 such that the rod 126 can only be rotated and/or slid axially relative to the coupling member 141 but otherwise cannot move relative to the coupling member 141 (for example, radially and/or circumferentially). In this way, whenever the threaded rod 126 is rotated (for example, by a physician), the threaded rod 126 can rotate relative to both the coupling member 141 and the proximal post 124.

In some examples, as depicted, the head portion 131 of the threaded rod 126 can include a first (or distal) flange 133, a second (or proximal) flange 135, and a head 137 extending proximally from the second flange 135. In some examples, the head portion 131 can include a greater or lesser number of flanges. The head 137 can be an engageable structure having one or more facets 137a. In some instances, as depicted, the facets 137a can define a rectangular projection. In some instances, the facets 137a of the head 137 can define projections having any of various shapes (for example, square, oval, square-oval, triangular, L-shaped, T-shaped, C-shaped, etc.). As introduced above, the head portion 131 can prevent the proximal post 124 and the coupling member 141 from moving proximally relative to the threaded rod 126 and can apply a distally directed force to the coupling member 141 and the proximal post 124 (for example, when radially expanding the prosthetic device 100). Specifically, the first flange 133 can have a width greater than a diameter of the inner bore 176 of the coupling member 141 to prevent the head portion 131 from moving into the inner bore 176 of the coupling member 141. Additionally, a distal surface of the first flange 133 can abut a proximal face of the coupling member 141 to apply the distally directed force to the coupling member 141. Due to the surface at the distal end 168d of the base 168 abutting the proximal face 173 of the proximal post 124, the coupling member 141 can likewise apply the distally directed force to the proximal post 124.

As introduced above, the head portion 131 of each of the threaded rods 126 can be releasably coupled to a corresponding actuation assembly of a delivery apparatus. In some examples, one or more other portions of the frame 102 (for example, the coupling member 141, etc.) can also releasably couple the prosthetic device 100 to an actuation assembly of a delivery apparatus. Referring to FIG. 3, an exemplary delivery apparatus 200 for delivering a prosthetic device or valve 202 (which may have the configuration of for example, the prosthetic device 100 or another configuration) to a desired implantation location is illustrated therein. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts. It will be appreciated the prosthetic valve 202 can have the configuration of the prosthetic device 100 shown in FIG. 1A or another configuration. For example, the prosthetic valve 202 can have a configuration similar those described in U.S. Provisional Patent Application Nos. 63/085,947 and/or 63/277,959, each of which is incorporated by reference herein.

The delivery apparatus 200 in the illustrated example generally includes a handle 204, a first elongated shaft 206 (which comprises an outer shaft in the illustrated example) extending distally from the handle 204, at least one actuation assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuation assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuation assemblies 208 for purposes of illustration, it should be understood that one actuation assembly 208 can be provided for each actuator (for example, actuator or threaded rod 126) on the prosthetic valve. For example, three actuation assemblies 208 can be provided for a prosthetic valve having three actuators. In some examples, a greater or fewer number of actuation assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery thereof to the implantation site.

The actuation assemblies 208 can be releasably coupled to the prosthetic valve 202. In the illustrated example, each actuation assembly 208 can be coupled to a respective actuator of the prosthetic valve 202 (such as being coupled to for example, the head portion 131 of the threaded rod 126, the coupling member 141, and/or other portions of the frame 102). Each actuation assembly 208 can comprise a support tube and an actuator member. When actuated, the actuation assembly 208 can transmit pushing and/or pulling forces to portions of the prosthetic valve to radially expand and collapse the prosthetic valve as previously described. The actuation assemblies 208 can be at least partially disposed radially within, and extend axially through, one or more lumens of the first shaft 206. For example, the actuation assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

In some examples, the first knob 211 can be a rotatable knob configured to produce axial movement of the first shaft 206 relative to the prosthetic valve 202 in the distal and/or proximal directions in order to deploy or deliver the prosthetic valve 202 from the delivery sheath 216 once the prosthetic valve 202 has been advanced to a location at or adjacent the desired implantation location with the patient's body. For example, rotation of the first knob 211 in a first direction (for example, clockwise) can result in retraction of the sheath 216 proximally relative to the prosthetic valve 202 and rotation of the first knob 211 in a second direction (for example, counter-clockwise) can cause advancement of the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuation assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can cause, produce, and/or result in rotation of the threaded rods of the prosthetic valve 202 via the actuation assemblies 208, as will be described in greater detail below with reference to FIGS. 4A-5. In some examples, rotation of the second knob 212 in a first direction (for example, clockwise) can radially expand the prosthetic valve 202 and rotation of the second knob 212 in a second direction (for example, counterclockwise) can radially collapse the prosthetic valve 202. In some examples, the second knob 212 can be actuated by sliding or moving the knob 212 axially, such as pulling and/or pushing the knob.

The third knob 214 can be a rotatable knob operatively connected to a proximal end portion of each actuation assembly 208. The third knob 214 can be configured to retract an outer sleeve or support tube of each actuation assembly 208 to disconnect the actuation assemblies 208 from the coupling members 141 of the prosthetic valve 202. Once the actuation assemblies 208 are uncoupled from the prosthetic valve 202, the delivery apparatus 200 can be removed from the patient, leaving just the prosthetic valve 202 in the patient.

Turning now to FIGS. 4A-5, an exemplary actuation assembly 300 of a delivery apparatus configured to be releasably coupled to the frame 102 for actuation thereof is shown and described. The actuation assembly 300 can be, for example, one of the actuation assemblies 208 of the delivery apparatus 200 shown in FIG. 2. The frame 102 can be utilized in the prosthetic valve 100 shown in FIG. 1A or other prosthetic valves.

The actuation assembly 300 can include two concentric or coaxial components, namely an outer sleeve 302 and an inner driver 304. In some instances, the outer sleeve 302 can be integrally formed as a single, unitary component and the inner driver 304 can be integrally formed as a single, unitary component. In some instances, as depicted, the locking sleeve 302 and the inner driver 304 can each comprise one or more segments that are formed as separate components that are coupled together (for example, via fasteners, adhesive, mating features, and/or other means for coupling). For example, the outer sleeve 302 (also referred to herein as a “sleeve member”) can comprise a sleeve shaft 306 and a sleeve head 308 connected at a distal end of the sleeve shaft 306. Additionally, the driver 304 (also referred to herein as “driver member” or “actuation member”) can include a driver shaft 310 and a driver head 312 connected at a distal end of the driver shaft 310. The driver shaft 310 can also be referred to as an “actuation shaft” and the driver head 312 can also be referred to as an “actuation head” or “engagement head.” In some examples, as depicted, the driver 304 can also include a cover 314 which circumferentially surrounds a distal end portion of the driver head 312. In some examples, the cover 314 and the driver head 312 can be integrally formed as a single, unitary component.

FIG. 4A illustrates the actuation assembly 300 releasably coupled to or engaged with one of the threaded rods 126, with the outer sleeve 302 removed for illustration purposes. Specifically, in FIG. 4A, the driver 304 is releasably coupled to or engaged with a head portion 131 of the threaded rod 126. The cover 314 is shown transparently in FIG. 4A for purposes of illustration. FIG. 4B and the cross-sectional view of FIG. 5A illustrate the actuation assembly 300 releasably coupled to or engaged with the frame 102. Specifically, the driver 304 is releasably coupled to or engaged with the head portion 131 of the threaded rod 126. Additionally, the sleeve head 308 of the outer sleeve 302 is releasably coupled to or engaged with the coupling member 141. The sleeve head 308 is shown transparently in FIG. 4B for purposes of illustration. FIG. 5B illustrates a distal end of the actuation assembly 300 without a threaded rod 126 coupled thereto.

The sleeve shaft 306 and the driver shaft 310 can each comprise an elongated flexible shaft having any of various constructions and can be made from any of various materials. For example, the sleeve shaft 306 and/or the driver shaft 310 can comprise a flexible shaft comprising one of more polymeric layers and optionally one or more reinforcing layers embedded within or surrounding one or more the polymeric layers. The reinforcing layers can comprise a braided layer and/or a layer comprising a helical coil. In some examples, as depicted in FIG. 5A, the sleeve shaft 306 can comprise one or more segments that are formed as separate components that are coupled together (for example, via fasteners, adhesive, mating features, and/or other means for coupling). In some examples, the sleeve shaft 306 can be integrally formed as a single, unitary component. In some examples, the driver shaft 310 can comprise a cable, wire, or flexible rod.

As shown in FIG. 5A, the driver shaft 310 can be coaxial with and disposed within a lumen of the sleeve shaft 306. The driver shaft 310 can be rotatable relative to the sleeve shaft 306. Additionally, the driver head 312 can be coaxial with and at least partially disposed within a channel of the sleeve head 308. The driver head 312 can be rotatable relative to the sleeve head 308.

In the illustrated example, the driver head 312 includes a distal engagement portion 316 having a socket 323 (FIG. 5B) configured to engage with the head portion 131 of the threaded rod 126 and a proximal portion 318 coupled to the driver shaft 310 (for example, fixedly coupled, etc.). In particular, the engagement portion 316 of the driver head 312 can include one or more distal extensions 320. In the illustrated example, the engagement portion 316 includes two extensions 320 that extend distally from a distal end thereof and are evenly spaced apart in a circumferential direction. The extensions 320 can include inner surfaces 321 having a complementary configuration to the head 137 of the threaded rod 126. For example, as shown in FIG. 5B, the inner surfaces 321 of the extensions 320 can define a socket 323 configured to releasably mate with the facets 137a of the head 137. In some examples, as depicted, an inner surface of the cover 314 can also partially define the socket 323 (for example, when the extensions 320 are circumferentially spaced apart, etc.). When the driver head 312 is fully engaged with the head portion 131 of the threaded rod 126, the socket 323 (for example, defined by the extensions 320 and the cover 314, etc.) fits over the head 137 and a distal end of the extensions 320 and a distal end of the cover 314 can be aligned with or abut a proximal face of the flange 135. In this way, when fully engaged, rotation of the driver 304 can cause rotation of the threaded rod 126.

The distal engagement portion 316 can have a smaller outer diameter than the proximal portion 318, such that the driver head 312 has an overall stepped configuration. In some examples, as depicted, the cover 314 can be positioned around the distal engagement portion 316 and can abut a shoulder or step 317 between the distal and proximal portions 316, 318 of the driver head 312. In the illustrated example, the outer diameter of the cover 314 and the outer diameter of the proximal portion 318 can be equal, and can be different sizes in some examples. In some instances, the driver head 312 and the cover 314 are integrally formed as a single, unitary component.

The proximal portion 318 of the driver head 312 can include a bore 322 (for example, a tapered bore, a straight bore, etc.) configured to receive and retain the driver shaft 310 (FIG. 5). The driver shaft 310 and the driver head 312 can be coupled together in a fixed manner, such that rotation of the driver shaft 310 causes rotation of the driver head 312. In some examples, as depicted in FIGS. 4A-4B, an outer wall of the proximal portion 318 can include an opening 324 and the cover 314 can include a slot 326.

As described above, the driver head 312 can be positioned within a channel defined by an inner wall 328 of the sleeve head 308. A distal end portion 330 of the sleeve head 308 can include internal threads configured to engage with external threads of the frame 102, specifically the external threads of the threaded engagement portion 172 of the coupling member 141. When the sleeve head 308 is fully engaged with the coupling member 141, as depicted in FIGS. 4B and 5A, based on a threaded connection between the distal end portion 330 and the threaded engagement portion 172, the distal end portion 330 of the sleeve head 308 can abut the shoulder 169 of the coupling member 141. A proximal end portion 332 of the sleeve head 308 can be coupled to sleeve shaft 306 (for example, a distal end of the sleeve shaft 306), such that the sleeve head 308 is fixed relative to the sleeve shaft 306. For example, rotation of the sleeve shaft 306 can cause rotation of the sleeve head 308.

In examples, the respective engagements of the sleeve head 308 and the driver head 312 with the coupling member 141 and the head portion 131 described above can prevent or limit unwanted movement and/or rotation of the prosthetic valve during radial expansion and/or radial compression thereof, thereby decreasing unwanted movement of the prosthetic valve (such as for example, after placement or positioning within the native valve).

For example, the threaded connection between the sleeve head 308 and the coupling member 141 as well as the fitting or bracing of the coupling member 141 relative to the proximal post 124 can limit unwanted lateral movement and/or rotational displacement of the frame during actuation (such as for example, unwanted lateral movement and/or rotational displacement along Y and Z axes, wherein the Y and Z axes are perpendicular to the longitudinal axis of the actuation assembly 300). For example, the engagement of the pair of arms 180 at the proximal end 110 of the proximal post 124 with the base 168 of the coupling member 141 as well as the engagement of the extensions 170 of the coupling member 141 with the proximal end 110 of the proximal post 124 can counter-act rotational forces applied to the frame 102 by the threaded rods 126 during for example, expansion of the frame. In the absence of a counterforce acting against such rotational forces, the frame can tend to “jerk” or rock in the direction of rotation of the rods when they are actuated to expand the frame. The illustrated configuration is advantageous in that the sleeve head 308, when engaging the threaded engagement portion 172 of the coupling member 141, and the coupling member 141, when engaging the proximal posts 124 of the frame 102, can prevent or mitigate such jerking or rocking motion of the frame 102 when the frame is radially expanded.

In some examples, the threaded connection between threaded engagement portion 172 of the coupling member 141 and the distal end portion 330 of the sleeve head 308 can maintain the engaged position of the actuation assembly 300 with the frame 102. Specifically, this threaded connection can maintain the engaged position of the driver 304 with the head portion 131 of the threaded member 126. In this way, the threaded connection between the coupling member 141 and the sleeve head 308 can limit unwanted axial movement between the actuation assembly 300 and the frame 102 (such as for example, unwanted axial movement the driver 304 relative to the head portion 131).

In some examples, engagement (contact) between the socket 323 of the driver 304 (for example, inner surfaces 321 of the extensions 320 and the inner surface of the cover 314) and the complementary facets 137a on the exterior surface of the head 137, can stably maintain the engagement or coupling between the head portion 131 and the socket 323 of the driver 304 during rotation of the threaded member 126 to limit unwanted lateral movement and/or rotational displacement of the frame during actuation that might otherwise result from disengagement or partial disengagement between the actuator and the head of the threaded member.

As introduced above, proximal (opposing) end portions of both of the outer sleeve shaft 306 and the driver shaft 310 can be operatively coupled to a handle or other controls of a delivery apparatus to enable the physician to control rotation of the sleeve shaft 306 and the sleeve head 308 coupled at the distal end thereof and/or to control rotation of the driver shaft 310 and the driver head 312 coupled at the distal end thereof. For example, FIGS. 6-8 illustrate a delivery apparatus 400 including the actuation assembly 300 that is engaged with and/or coupled to the frame 102 and a handle 402 operatively coupled to the actuation assembly 300. The frame 102 can be utilized in the prosthetic valve 100 shown in FIG. 1A or other prosthetic valves. Although only one actuation assembly 300 is coupled to the frame 102 in the illustrated example, in some examples, one or more additional actuation assemblies 300 can also be coupled to the frame 102 and the handle 402. In FIG. 6, the proximal end portion of the sleeve member 302 is broken away for purposes of illustration.

The handle 402 can include a first knob 404 and a second knob 406. Although not shown in FIGS. 6-8, in some examples, the handle 402 can also include one or more additional knobs (for example, similar to knob 211 of handle 204). The delivery apparatus 400 can include other components, such as an outer shaft or delivery sheath (for example, similar to delivery sheath 216 of handle 204) configured to house the prosthetic valve in its radially compressed, delivery state during delivery of the prosthetic valve through the patient's vasculature. As shown, the handle 402 can include a housing 410 with various compartments to hold the components of a first gear train 412 and a second gear train 414. In some examples, as depicted, the housing 410 does not fully encase the components of the first and second gear trains 412, 414. In some examples, the housing 410 can fully encase or surround the components and can form a gripping portion for a user to hold the handle in their hand.

In the illustrated example, the first gear train 412 can be operatively coupled to the first knob 404, which is configured to rotate all of the sleeve members 302 together to connect or disconnect the sleeve members 302 from the frame 102 upon rotation of the knob 404. For example, the output shafts of the first gear train 412 can be coupled to the sleeve shafts 306 such that operation of the first gear train 412 results in rotation of the sleeve heads 308 relative to the coupling members 141 of the frame 102.

The second gear train 414 can be operatively coupled to the second knob 406, which is configured to rotate all of the driver members 304 together upon rotation of the knob 406 to control radial expansion or compression of the frame 102 of the prosthetic valve. For example, the output shafts of the second gear train 414 can be coupled to the driver members 304 such that operation of the second gear train 414 results in rotation of the driver members 304 and consequently rotation of the actuators 106 (for example, the threaded rods 126, etc.) of the frame 102. In one example, the delivery apparatus 400 can include a counter-rotation mechanism where the second gear train 414 is configured to rotate two sets of actuator drivers in opposite directions.

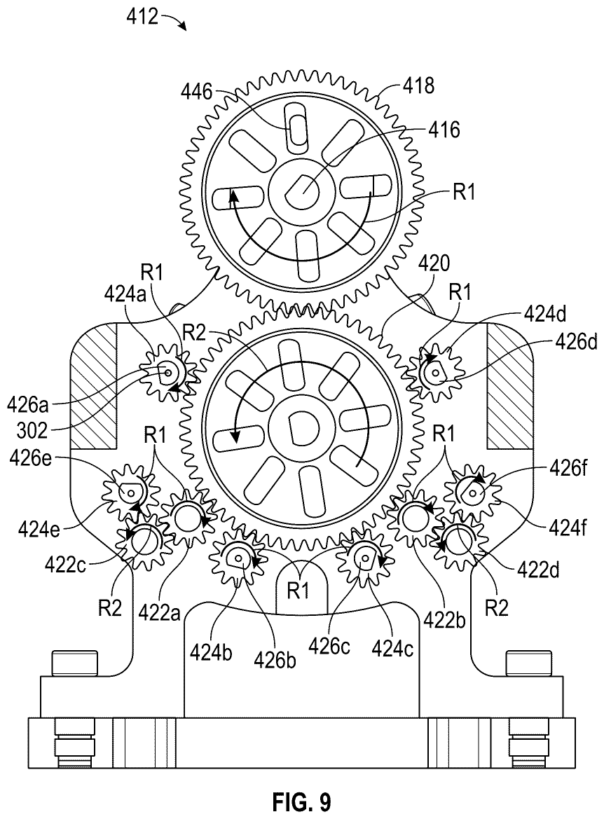

FIG. 9 illustrates one implementation of the first gear train 412. In the example, the first gear train 412 includes an input shaft 416 and an input gear 418 coupled to the input shaft 416. The input shaft 416 can be coupled to the first knob 404 of the handle 402, such that rotation of the first knob 404 results in rotation of the input shaft 416. The input gear 418 rotates with the input shaft 416. The rotational direction R1 of the input gear 418 can be clockwise or counterclockwise, depending on the direction in which the first knob 404 is rotated. In FIG. 9, the rotational direction R1 is depicted as clockwise.

The first gear train 412 can include a first idler gear 420 having teeth that are meshed with the teeth of the input gear 418, such that rotation of the input gear 418 drives the first idler gear 420. In one example, rotation of the input gear 418 in a first direction R1 drives the first idler gear 420 in a second direction R2 that is opposite to the first direction (whether R2 is clockwise or counterclockwise will depend on the rotational direction R1 as determined by the rotation of the first knob 404). In FIG. 9, the rotational direction R2 is depicted as counterclockwise.

The first gear train 412 can include a set of second idler gears 422a, 422b, 422c, and 422d. The teeth of the second idler gears 422a, 422b can be meshed with the teeth of the first idler gear 420, such that rotation of the first idler gear 420 drives the second idler gears 422a, 422b. In one example, rotation of the first idler gear 420 in the second direction R2 drives the second idler gears 422a, 422b in the first direction R1. The teeth of the other idler gears 422c, 422d can be meshed teeth of the idler gears 422a, 422b, respectively. The second idler gears 422c, 422d rotate in the same direction R2 as the first idler gear 420.

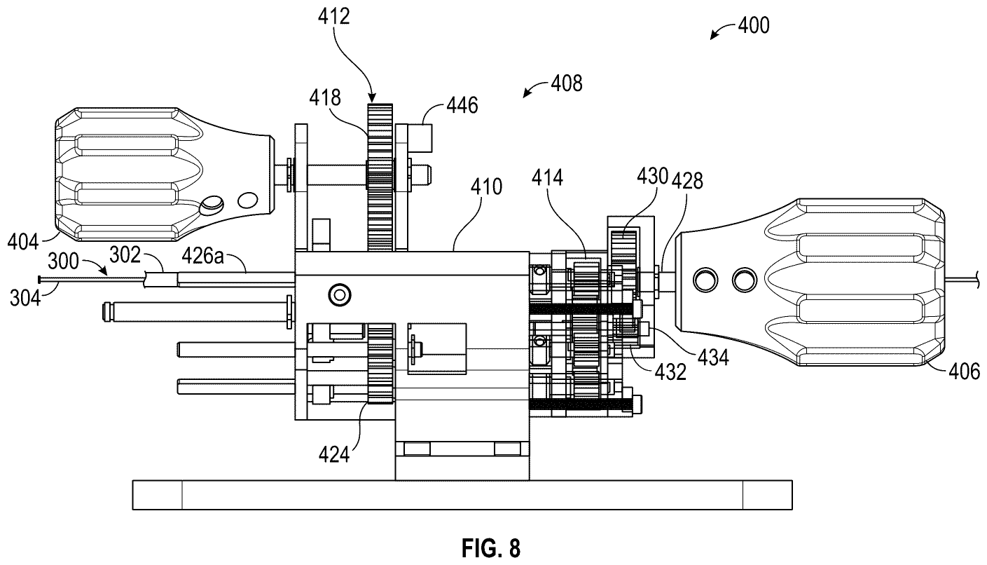

The first gear train 412 can include a set of first output gears (which can also be referred to as “sleeve gears”) angularly spaced apart about a central axis of the first idler gear 420 and having teeth meshed with the teeth of the first idler gear 420. In the example, the set of first output gears includes output gears 424a, 424b, 424c, 424d. The output gears 424a, 424b, 424c, 424d rotate in a direction R1 that is opposite to the direction R2 in which the first idler gear 420 is rotating. In one example, the output gears 424a, 424b, 424c, 424d arc coupled to output shafts 426a, 426b, 426c, 426d, respectively. The output shafts 426a, 426b, 426c, 426d can be coupled to a first set of sleeve members (for example, sleeve members 302). As best shown in FIG. 8, in the illustrated example, the output shaft 426a is shown as being coupled to a sleeve member 302 of the actuation assembly 300. Although not shown, the other output shafts 426 (which are omitted for purposes of illustration) can be similarly coupled to respective sleeve members 302 of the delivery apparatus.

The first gear train 412 can include a second set of output gears (which can also be referred to as “sleeve gears”) having teeth meshed with the teeth of the second idler gears 422c, 422d. In the example, the second set of output gears includes output gears 424c, 424f. The output gears 424e, 424f rotate in a direction R1 that is opposite to the direction R2 in which the second idler gears 422c, 422d are rotating. As such, the output gears 424c, 424f of the second set of output gears rotate in a direction that is the same as the direction in which the output gears 424a, 424b, 424c, 424d of the first set of output gears rotate. In one example, the output gears 424e, 424f are coupled to output shafts 426e, 426f, respectively. The output shafts 426e, 426f can be coupled to a second set of sleeve members (for example, sleeve members 302).

The first and second sets of output gears 424a, 424b, 424c, 424d, 424c, 424f can be collectively referred to herein as output gears 424 or sleeve gears 424. The output shafts 426a, 426b, 426c, 426d, 426e, 426f can be collectively referred to herein as output shafts 426.

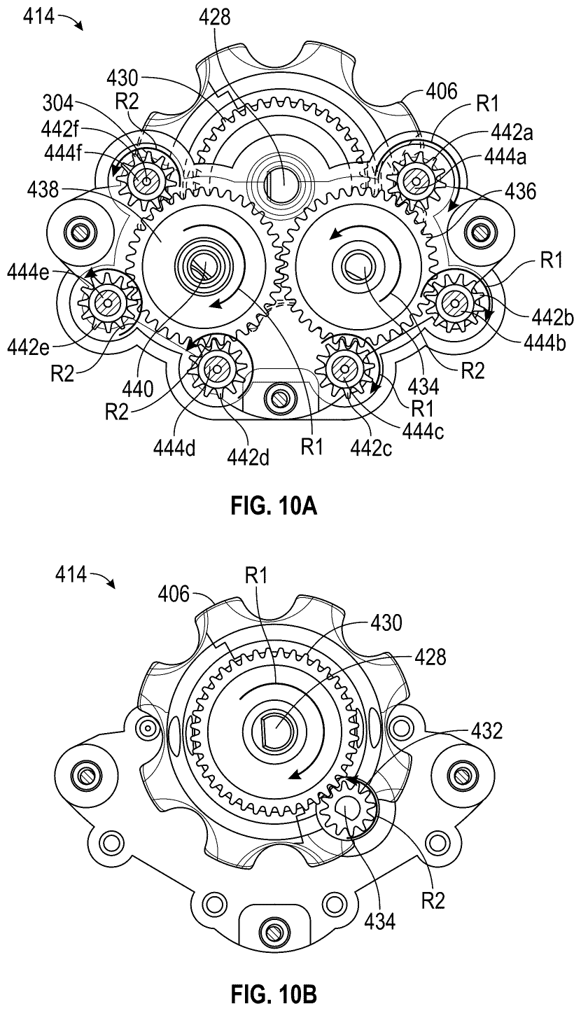

FIGS. 10A-10B illustrate one implementation of the second gear train 414. In the example, the second gear train 414 includes an input shaft 428 and an input gear 430 coupled to the input shaft 428. The input shaft 428 can be coupled to the second knob 406 of the handle 402, such that rotation of the second knob 406 results in rotation of the input shaft 428. The input gear 430 rotates with the input shaft 428. The rotational direction R1 of the input gear 430 can be clockwise or counterclockwise, depending on the direction in which the second knob 406 is rotated. In FIGS. 10A-10B, the rotational direction R1 is depicted as clockwise.

The second gear train 414 can include a transmission gear 432 coupled to a transmission shaft 434, which can be arranged in parallel to the input shaft 428. The teeth of the input gear 430 are meshed with the teeth of the transmission gear 432 such that rotation of the input gear 430 drives the transmission gear 432. The transmission shaft 434 rotates with the transmission gear 432. In one example, rotation of the input gear 430 in a first direction R1 drives the transmission gear 432 in a second direction R2 that is opposite to the first direction (whether R2 is clockwise or counterclockwise will depend on the rotational direction R1 as determined by the rotation of the second knob 406). In FIGS. 10A-10B, the rotational direction R2 is depicted as counterclockwise.

The second gear train 414 can include a first driving gear 436 coupled to the transmission shaft 434 and disposed distally to the transmission gear 432. In this case, rotation of the transmission shaft 434 in response to driving the transmission gear 432 by the input gear 430 is translated to rotation of the first driving gear 436. The first driving gear 436 rotates in the same direction R2 as the transmission gear 432.

The second gear train 414 can include a second driving gear 438 supported on a driving shaft 440 that is arranged in parallel to the transmission shaft 434. The teeth of the second driving gear 438 are meshed with the teeth of the first driving gear 436 such that rotation of the first driving gear 436 drives the second driving gear 438. The driving shaft 440 rotates with the second driving gear 438. The second driving gear 438 rotates in a direction R1 that is opposite to the direction R2 in which the first driving gear 436 rotates.

The second gear train 414 can include a set of first output gears (which can also be referred to as “pinion gears”) angularly spaced apart about a central axis of the first driving gear 436 and having teeth meshed with the teeth of the first driving gear 436. In the example, the set of first output gears includes output gears 442a, 442b, 442c. The output gears 442a, 442b, 442c rotate in a direction R1 that is opposite to the direction R2 in which the first driving gear 436 is rotating. In one example, the output gears 442a, 442b, 442c are coupled to output shafts 444a, 444b, 444c, respectively. The output shafts 444a, 444b, 444c can be coupled to a first set of driver members (for example, driver members 304).

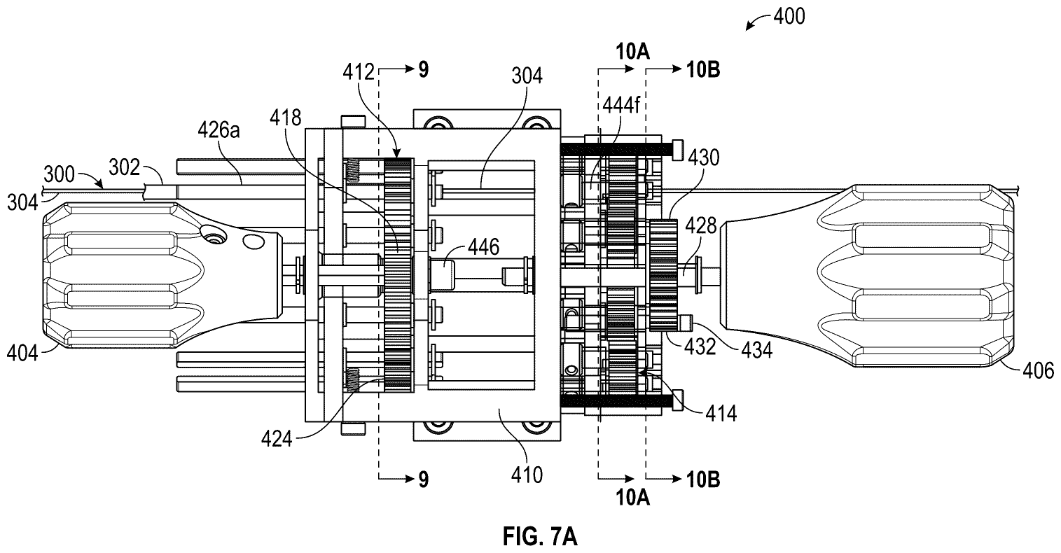

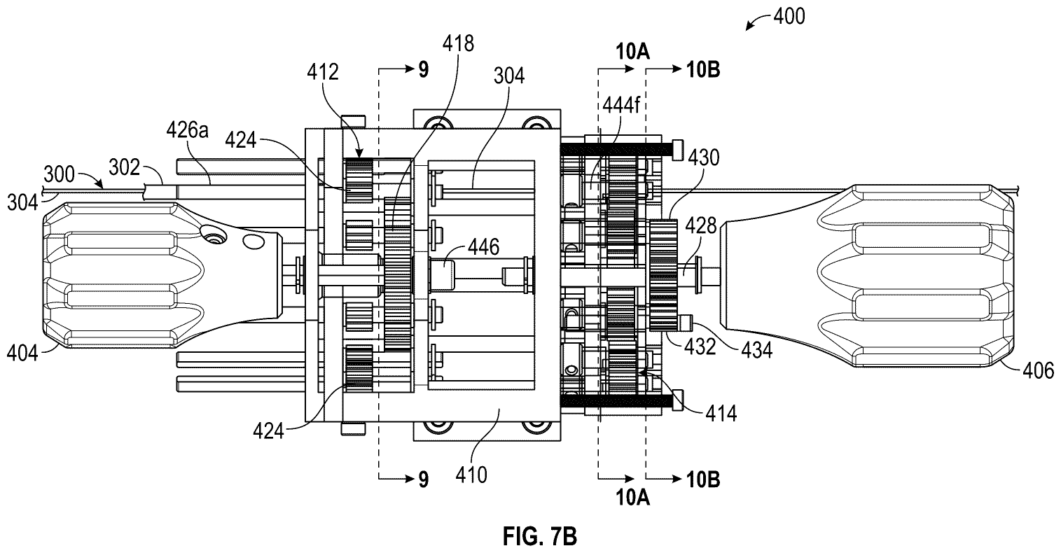

The second gear train 414 can include a second set of output gears (which can also be referred to as “pinion gears”) angularly spaced apart about a central axis of the second driving gear 438 and having teeth meshed with the teeth of the second driving gear 438. In the example, the second set of output gears includes output gears 442d, 442c, 442f. The output gears 442d, 442c, 442f rotate in a direction R2 that is opposite to the direction R1 in which the second driving gear 438 is rotating. As such, the output gears 442d, 442c, 442f of the second set of output gears rotate in a direction that is opposite to the direction in which the output gears 442a, 442b, 442c of the first set of output gears rotate. In one example, the output gears 442d, 442e, 442f are coupled to output shafts 444d, 444c, 444f, respectively. The output shafts 444d, 444c, 444f can be coupled to a second set of driver members (for example, driver members 304). As best shown in FIGS. 7A-7B, in the illustrated example, the output shaft 444f is shown as being coupled to a driver member 304 of the actuation assembly 300. The other output shafts 444 (which are omitted for purposes of illustration) can be similarly coupled to respective driver members 304 of the delivery apparatus. Each driver member 304 can extend distally from a respective output shaft 444 through a respective output shaft 426 of a sleeve gear 424 and a respective sleeve member 302.

In some examples, the delivery apparatus 400 can include one or more mechanisms configured to selectively transition the first gear train 412 between a first configuration and a second configuration. In the first configuration, rotation of the first knob 404 enables rotation of the gears of the first gear train 412, thereby rotating the sleeve members 302 of the actuation assembly 300. During assembly of the prosthetic valve to the actuation assemblies, the first gear train 412 can be in the first configuration to enable the sleeve member 302 to be threadedly engaged with the coupling member 141, such that the actuation assembly 300 can be coupled and/or attached to the frame 102. Once assembled, the first gear train 412 can be transitioned to the second configuration. In the second configuration, rotation of the gears of the first gear train 412, the first knob 404, and/or the sleeve members 302 of the actuation assembly 300 can be prevented. Preventing such rotation can maintain the actuation assemblies 300 in the engaged position relative to the frame 102 and can limit unwanted axial movement between the actuation assembly 300 and the frame 102 (such as for example, unwanted axial movement the driver 304 relative to the head portion 131), for example, while the prosthetic valve is delivered to an implantation site and expanded. When expansion is complete, the first gear train 412 can be transitioned back to the first configuration and the gears of the first gear train 412 can be rotated (for example, by the first knob 404) in an opposite direction, to disengage the sleeve member 302 from the coupling member 141 of the frame 102.

In some examples, as depicted, the handle 402 can include a locking pin 446 configured to selectively transition the first gear train 412 between the first configuration and the second configuration. For example, the locking pin 446 can be configured to selectively lock and unlock one or more gears of the first gear train 412. Specifically, in a locked position (FIG. 9), the locking pin 446 can extend axially through an opening of the input gear 418, such that rotation of the input gear 418 is prevented or limited. The locking pin 446 can be at least partially removed from the locked position (for example, by unscrewing the locking pin 446, by applying an axial force to the locking pin 446 to slide the pin 446 away from the gear 418, etc.) such that the locking pin 446 is in an unlocked position. In the unlocked position, the locking pin 446 does not extend axially through the opening of the input gear 418, such that the first gear train 412 can freely rotate in response to rotation of the first knob 404. In some examples, the locking pin 446 can be configured to selectively pass through openings of other gears in the first gear train 412 (for example, the first idler gear 420, etc.).

Alternatively or in addition to the locking pin 446, the first gear train 412 can be transitioned between the first and second configurations described above by disengaging one or more gears from the first gear train 412. For example, as shown in FIGS. 7A-7B, the sleeve gears 424 can be configured to be selectively disengaged from the first gear train 412, such that the teeth of the sleeve gears 424 are no longer meshed with the teeth of other gears of the first gear train 412. In particular, the sleeve gears 424 can move or slide axially along the output shafts 426 between an engaged position (FIG. 7A) and a disengaged position (FIG. 7B). When the sleeve gears 424 are in the disengaged position, the first knob 404 and the other gears of the first gear train 412 (for example, the input gear 418, the first idler gear 420, the second idler gears 422a, 422b, 422c, 422d, etc.) are permitted to rotate, however such rotation does not result in rotation of the sleeve gears 424 which are disengaged from the first gear train 412. When the sleeve gears 424 are in the engaged position, rotation of the first knob results in rotation of the sleeve gears 424 and the sleeves 302, as previously described. In some examples, other gears in the first gear train 412 (for example, the input gear 418, the first idler gear 420, etc.) can be configured to move or slide axially along their respective shaft to selectively disengage from the first gear train 412 and transition the first gear train 412 into the second configuration.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for implanting a prosthetic valve comprises: at least one actuation assembly configured to actuate an actuator member of a prosthetic valve, the actuator member configured to be rotated via rotation of a head thereof to radially expand or compress a frame the prosthetic valve, the actuation assembly comprising: a sleeve member having a lumen, wherein a distal end portion of the sleeve member comprises internal threads; a driver member coaxially disposed within the lumen of the sleeve member, the driver member being rotatable relative to the sleeve member, wherein a distal end portion of the driver member comprises a socket configured to receive the head of the actuator member; and wherein the actuation assembly is configured to be transitioned between an engaged position and a disengaged position with the frame.

Example 2. The delivery apparatus of any example herein, particularly example 1, wherein the actuation assembly is configured such that, in the engaged position, the internal threads of the sleeve member engage with external threads of the frame.

Example 3. The delivery apparatus of any example herein, particularly either example 1 or example 2, wherein the actuation assembly is configured such that, in the engaged position, the socket of the driver member engages with the head of the actuator member.

Example 4. The delivery apparatus of any example herein, particularly any one of examples 1-3, wherein the sleeve member comprises a sleeve shaft and a sleeve head disposed at a distal end of the sleeve shaft, wherein the sleeve head includes the internal threads.

Example 5. The delivery apparatus of any example herein, particularly example 4, wherein the actuation assembly is configured such that, in the engaged position, the sleeve head and the sleeve member limit lateral and/or rotational displacement of the frame during rotation of the driver member.

Example 6. The delivery apparatus of any example herein, particularly any one of examples 1-5, wherein an interior surface of the socket comprises one or more first facets, the one or more first facets having a complementary configuration to one or more second facets on an exterior surface of the head of the actuator member.

Example 7. The delivery apparatus of any example herein, particularly example 6, wherein the actuation assembly is configured such that, in the engaged position, the one or more first facets of the socket engage the one or more second facets of the head and enable, via rotation of the driver member relative to the sleeve member, transmission of torque to the head of the actuator member and rotation of the actuator member.

Example 8. The delivery apparatus of any example herein, particularly any one of examples 4-7, wherein the driver member comprises a driver shaft and a driver head disposed at a distal end of the driver shaft, wherein the driver head includes the socket.

Example 9. The delivery apparatus of any example herein, particularly example 8, wherein the driver head includes a cover at least partially defining the socket.

Example 10. The delivery apparatus of any example herein, particularly either example 8 or example 9, wherein the driver head is positioned within a lumen of the sleeve head.

Example 11. A delivery apparatus for a prosthetic valve, the delivery apparatus comprising: a handle; a driver having a proximal end portion coupled to the handle and a distal end portion comprising an engagement head to releasably couple the driver to an actuator of the prosthetic valve; and a sleeve member operatively coupled to the driver, the sleeve member having a proximal end portion coupled to the handle and a distal end portion comprising internal threads to releasably coupled the sleeve member to a frame of the prosthetic valve, wherein the driver extends through the sleeve member.

Example 12. The delivery apparatus of any example herein, particularly example 11, further comprising first and second gear trains disposed within or supported by the handle, wherein the first gear train operatively couples the sleeve member to a first knob for transferring rotation of the first knob to the sleeve member and wherein the second gear train operatively couples the driver to a second knob for transferring rotation of the second knob to the driver.

Example 13. The delivery apparatus of any example herein, particularly example 12, further comprising a locking pin configured to extend through a gear of the first gear train in a locked position to prevent rotation of the gear, thereby preventing the transfer of rotation from the first knob to the sleeve member.

Example 14. The delivery apparatus of any example herein, particularly either example 12 or example 13, wherein at least one gear of the first gear train is configured to transition between an engaged position relative to the first gear train and a disengaged position relative to the first gear train, thereby allowing the transfer of rotation from the first knob to the sleeve member in the engaged position and preventing such transfer in the disengaged position.

Example 15. The delivery apparatus of any example herein, particularly example 14, wherein the at least one gear is configured to move axially along a shaft between the engaged position and the disengaged position.

Example 16. The delivery apparatus of any example herein, particularly either example 14 or example 15, wherein the at least one gear is a sleeve gear coupled to the sleeve member.

Example 17. The delivery apparatus of any example herein, particularly any one of examples 11-16, wherein the engagement head comprises a socket corresponding to a head of the actuator.

Example 18. The delivery apparatus of any example herein, particularly any one of examples 11-17, wherein the driver is rotatable relative to the sleeve member.

Example 19. A prosthetic valve, comprising: a radially expandable and compressible frame comprising a plurality of interconnected struts, wherein the frame is radially expandable and compressible between a radially compressed state and a radially expanded state; a valvular structure disposed within the frame and configured to regulate the flow of blood through the frame in one direction; at least one rotatable actuator operatively coupled to the frame, wherein the actuator is rotatable relative to the frame in a first rotational direction to produce radial expansion of the frame from the radially compressed state to the radially expanded state; and at least one coupling member having external threads, wherein the coupling member is coupled to the actuator and the frame, wherein the coupling member is fixed relative to the frame in the first rotational direction.

Example 20. The prosthetic valve of any example herein, particularly example 19, wherein the actuator extends through an inner bore of the coupling member and an inner bore of the frame.

Example 21. The prosthetic valve of any example herein, particularly either example 19 or example 20, wherein a head of the actuator is proximal to the coupling member.

Example 22. The prosthetic valve of any example herein, particularly any one of examples 19-21, wherein the coupling member is coupled to a proximal end of the frame.

Example 23. The prosthetic valve of any example herein, particularly example 22, wherein the proximal end of the frame includes a plurality of arms.

Example 24. The prosthetic valve of any example herein, particularly example 23, wherein the coupling member is disposed between the plurality of arms.

Example 25. The prosthetic valve of any example herein, particularly either example 23 or example 24, wherein the coupling member comprises distal extensions extending axially along a length of the frame.

Example 26. The prosthetic valve of any example herein, particularly example 25, wherein the distal extensions of the coupling member are circumferentially spaced apart and are positioned between the plurality of arms, thereby preventing rotational movement of the coupling member relative to the frame.

Example 27. The prosthetic valve of any example herein, particularly example 26, wherein the coupling member includes one or more facets, the one or more facets having a complementary configuration to inner surfaces of the plurality of arms of the frame.

Example 28. The prosthetic valve of any example herein, particularly any one of examples 19-27, wherein the actuator is rotatable relative to the frame in a second rotational direction to produce radial compression of the frame from the radially expanded state to the radially compressed state; and wherein the coupling member is fixed relative to the frame in the second rotational direction.

Example 29. An assembly comprising: a prosthetic valve having a radially expandable and compressible frame, a valvular structure disposed within the frame and configured to regulate the flow of blood through the frame in one direction at least one actuator operatively coupled to the frame, and a threaded coupling member, wherein the actuator is configured to be rotated, via rotation of a head thereof, to radially expand or compress the frame; and a delivery device comprising a sleeve member having internal threads and a driver member at least partially disposed within the sleeve member, wherein the sleeve member is configured to form a releasable threaded connection with the coupling member and the driver member is configured to releasably engage the actuator and rotate the actuator upon rotation of the driver member.

Example 30. The assembly of any example herein, particularly example 29, wherein the coupling member comprises external threads configured to engage with the internal threads of the sleeve member.

Example 31. The assembly of any example herein, particularly either example 29 or example 30, wherein the coupling member is integrally formed with the frame.

Example 32. The assembly of any example herein, particularly example 30, wherein the coupling member comprises a base, extensions extending axially from a distal end of the base, and a threaded engagement portion proximal to the base, the threaded engagement portion including the external threads.

Example 33. The assembly of any example herein, particularly example 32, wherein a proximal end of the frame is disposed between the extensions.

Example 34. The assembly of any example herein, particularly example 33, wherein the frame comprises arms extending axially from the proximal end, wherein the base of the coupling member is disposed between the arms.

Example 35. The assembly of any example herein, particularly example 34, wherein the coupling member comprises facets on exterior surfaces of the base and the threaded engagement portion, wherein the facets are aligned with inner surfaces of the arms.

Example 36. The assembly of any example herein, particularly any one of examples 32-35, wherein a distal surface of the base abuts a proximal surface of the frame.

Example 37. The assembly of any example herein, particularly any one of examples 29-36, wherein the coupling member is fixed relative to the frame in a rotational direction.

Example 38. A method of implanting a prosthetic valve, comprising: engaging internal threads of a sleeve coupled to a handle with external threads of a frame of the prosthetic valve; engaging a socket of a driver coupled to the handle with a head of an actuator of the frame; delivering the prosthetic valve to an implantation location within a patient's body; radially expanding the prosthetic valve to a functional size; and releasing the sleeve from the frame and releasing the driver from the actuator.

Example 39. The method of any example herein, particularly example 38, wherein radially expanding the prosthetic valve comprises rotating a first knob operatively coupled to the driver, thereby rotating the driver relative to the sleeve.

Example 40. The method of any example herein, particularly either example 38 or example 39, wherein engaging the internal threads of the sleeve with the external threads of the frame comprises rotating a second knob operatively coupled to the sleeve in a first direction; and wherein releasing the sleeve from the frame comprises rotating the second knob in a second direction.

Example 41. A method of implanting a prosthetic valve, comprising: engaging an end portion of a sleeve coupled to a handle with an end portion of a frame of the prosthetic valve; preventing rotation of an output gear of a gear train disposed within the handle and operatively coupled to the sleeve; delivering the prosthetic valve to an implantation location within a patient's body; radially expanding the prosthetic valve to a functional size; then allowing rotation of the output gear; and releasing the end portion of the sleeve from the end portion of the frame.

Example 42. The method of any example herein, particularly example 41, wherein radially expanding the prosthetic valve comprises rotating a driver relative to the sleeve, the driver extending through the sleeve and engaged with an end portion of an actuator of the frame.

Example 43. The method of any example herein, particularly either example 41 or example 42, wherein engaging the end portion of the sleeve with the end portion of the frame comprises rotating a knob operatively coupled to the gear train in a first direction; and wherein releasing the end portion of the sleeve from the end portion of the frame comprises rotating the knob in a second direction.

Example 44. The method of any example herein, particularly any one of examples 41-43, wherein preventing rotation of the output gear comprises disengaging a first gear from the gear train; and wherein allowing rotation of the output gear comprises meshing the first gear with the gear train.

Example 45. The method of any example herein, particularly example 44, wherein disengaging the first gear comprises moving the first gear along a shaft in a first axial direction away from the gear train; and wherein engaging the first gear comprises moving the first gear along the shaft in a second axial direction towards the gear train.

Example 46. The method of any example herein, particularly either example 44 or example 45, wherein the first gear comprises the output gear.

Example 47. The method of any example herein, particularly any one of examples 41-46, wherein preventing rotation of the output gear comprises positioning a locking pin within an opening of at least one gear of the gear train.

Example 48. An assembly comprising: a prosthetic heart valve comprising a frame and at least one actuator coupled to the frame, the frame comprising a threaded engagement portion having external threads, the actuator operable to move the frame between a radially expanded configuration and a radially compressed configuration; at least one actuation assembly comprising: an actuator driver configured to releasably engage the at least one actuator; and a sleeve member operatively coupled to the actuator driver and movable between a first position in which the actuator driver is retained in engagement with the at least one actuator and a second position in which the actuator driver is released from engagement from the at least one actuator, the sleeve member comprising internal threads configured for threaded engagement with the external threads of the frame in the first position.

Example 49. The assembly of any example herein, particularly example 48, wherein the frame further comprises a coupling member having a lumen and coupled to the frame, wherein the at least one actuator extends through the lumen, wherein the coupling member includes the threaded engagement portion.

Example 50. The assembly of any example herein, particularly either example 48 or example 49, wherein the coupling member is retained between a pair of arms extending proximally from the frame such that the coupling member is non-rotatable relative to the frame.

Example 51. The assembly of any example herein, particularly any one of examples 48-50, wherein the sleeve member and the threaded engagement portion of the frame are threadedly engaged in the first position.

Example 52. The assembly of any example herein, particularly any one of examples 48-51, further comprising a handle coupled to the at least one actuation assembly, the handle defining a longitudinal axis.

Example 53. The assembly of any example herein, particularly example 52, further comprising a first knob rotatably mounted on the handle and operatively coupled to the sleeve member such that rotation of the first knob relative to the handle moves the sleeve member between the first and second positions.

Example 54. The assembly of any example herein, particularly either example 52 or example 53, further comprising a first gear train operatively coupled to the first knob and the sleeve member, wherein the first gear train includes an output gear coupled to the sleeve member.

Example 55. The assembly of any example herein, particularly example 54, further comprising an output shaft connected to the output gear and the sleeve member such that rotation of the output gear rotates the output shaft and the sleeve member.

Example 56. The assembly of any example herein, particularly example 55, wherein the output gear is configured to move along the longitudinal axis between an engaged position in which the output gear is meshed with other gears of the first gear train and a disengaged position in which the output gear is not meshed with the other gears such that the sleeve member is positioned in the first position.

Example 57. The assembly of any example herein, particularly any one of examples 54-56, further comprising a locking pin configured to selectively extend through a gear of the first gear train to prevent rotation of the gear, thereby preventing the sleeve member from moving between the first position and the second position.

Example 58. The assembly of any example herein, particularly any one of examples 52-57, further comprising a second knob rotatably mounted on the handle and operatively coupled to the actuator driver such that rotation of the second knob relative to the handle rotates the actuator driver and the at least one actuator to the frame between the radially expanded configuration and the radially compressed configuration.

Example 59. The assembly of any example herein, particularly any one of examples 48-58, wherein the actuator driver extends co-axially through the sleeve member.

Example 60. The assembly of any example herein, particularly any one of examples 48-59, wherein the prosthetic heart valve comprises a plurality of actuators and the at least one actuation assembly comprises a plurality of actuation assemblies.

Example 61. The assembly of any example herein, particularly example 60, further comprising a first knob and a first gear train comprising an input gear and a plurality of output gears operatively coupled to the input gear, wherein each output gear is coupled to a sleeve member of a respective actuation assembly such that rotation of the first knob produces simultaneous rotation of the output gears and the sleeve members.

Example 62. The assembly of any example herein, particularly example 61, further comprising a second knob and a second gear train comprising an input gear and a plurality of output gears operatively coupled to the input gear, wherein each output gear of the second gear train is coupled to an actuator driver of a respective actuation assembly such that rotation of the second knob produces simultaneous rotation of the output gears of the second gear train and the actuator drivers.

Example 63. The assembly of any example herein, particularly example 62, wherein each actuator driver extends through an output gear of the first gear train and a sleeve member.

Example 64. The delivery apparatus, prosthetic valve, or assembly of any example herein, particularly any of examples 1-63, wherein the delivery apparatus, prosthetic valve, or assembly is sterilized.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A delivery apparatus for implanting a prosthetic valve, the delivery apparatus comprising:
at least one actuation assembly configured to actuate an actuator member of a prosthetic valve, the actuator member configured to be rotated via rotation of a head thereof to radially expand or compress a frame the prosthetic valve, the actuation assembly comprising:
a sleeve member having a lumen, wherein a distal end portion of the sleeve member comprises internal threads; and
a driver member coaxially disposed within the lumen of the sleeve member, the driver member being rotatable relative to the sleeve member, wherein a distal end portion of the driver member comprises a socket configured to receive the head of the actuator member,
wherein the actuation assembly is configured to be transitioned between an engaged position and a disengaged position with the frame.

2. The delivery apparatus of claim 1, wherein the actuation assembly is configured such that, in the engaged position, the internal threads of the sleeve member engage with external threads of the frame.

3. The delivery apparatus of claim 1, wherein the actuation assembly is configured such that, in the engaged position, the socket of the driver member engages with the head of the actuator member.

4. The delivery apparatus of claim 1, wherein the sleeve member comprises a sleeve shaft and a sleeve head disposed at a distal end of the sleeve shaft, wherein the sleeve head includes the internal threads.

5. The delivery apparatus of claim 4, wherein the actuation assembly is configured such that, in the engaged position, the sleeve head and the sleeve member limit lateral and/or rotational displacement of the frame during rotation of the driver member.

6. The delivery apparatus of claim 1, wherein an interior surface of the socket comprises one or more first facets, the one or more first facets having a complementary configuration to one or more second facets on an exterior surface of the head of the actuator member.

7. The delivery apparatus of claim 6, wherein the actuation assembly is configured such that, in the engaged position, the one or more first facets of the socket engage the one or more second facets of the head and enable, via rotation of the driver member relative to the sleeve member, transmission of torque to the head of the actuator member and rotation of the actuator member.

8. The delivery apparatus of claim 4, wherein the driver member comprises a driver shaft and a driver head disposed at a distal end of the driver shaft, wherein the driver head includes the socket.

9. The delivery apparatus of claim 8, wherein the driver head includes a cover at least partially defining the socket.

10. The delivery apparatus of claim 8, wherein the driver head is positioned within a lumen of the sleeve head.

11. A prosthetic valve, comprising:
a radially expandable and compressible frame comprising a plurality of interconnected struts, wherein the frame is radially expandable and compressible between a radially compressed state and a radially expanded state;
a valvular structure disposed within the frame and configured to regulate flow of blood through the frame in one direction;
at least one rotatable actuator operatively coupled to the frame, wherein the actuator is rotatable relative to the frame in a first rotational direction to produce radial expansion of the frame from the radially compressed state to the radially expanded state; and
at least one coupling member having external threads, wherein the coupling member is coupled to the actuator and the frame, wherein the coupling member is fixed relative to the frame in the first rotational direction.

12. The prosthetic valve of claim 11, wherein the actuator extends through an inner bore of the coupling member and an inner bore of the frame.

13. The prosthetic valve of claim 11, wherein the coupling member is coupled to a proximal end of the frame.

14. The prosthetic valve of claim 13, wherein the proximal end of the frame includes a plurality of arms.

15. The prosthetic valve of claim 14, wherein the coupling member is disposed between the plurality of arms.

16. The prosthetic valve of claim 11, wherein the coupling member comprises distal extensions extending axially along a length of the frame.

17. An assembly comprising:
a prosthetic valve having a radially expandable and compressible frame, a valvular structure disposed within the frame and configured to regulate the flow of blood through the frame in one direction at least one actuator operatively coupled to the frame, and a threaded coupling member, wherein the actuator is configured to be rotated, via rotation of a head thereof, to radially expand or compress the frame; and
a delivery device comprising a sleeve member having internal threads and a driver member at least partially disposed within the sleeve member, wherein the sleeve member is configured to form a releasable threaded connection with the coupling member and the driver member is configured to releasably engage the actuator and rotate the actuator upon rotation of the driver member.

18. A method of implanting a prosthetic valve, comprising:
engaging an end portion of a sleeve coupled to a handle with an end portion of a frame of the prosthetic valve;
preventing rotation of an output gear of a gear train disposed within the handle and operatively coupled to the sleeve;
delivering the prosthetic valve to an implantation location within a patient's body;
radially expanding the prosthetic valve to a functional size; then allowing rotation of the output gear; and
releasing the end portion of the sleeve from the end portion of the frame.

19. The method of claim 18, wherein radially expanding the prosthetic valve comprises rotating a driver relative to the sleeve, the driver extending through the sleeve and engaged with an end portion of an actuator of the frame.

20. The method of claim 18, wherein preventing rotation of the output gear comprises disengaging a first gear from the gear train; and wherein allowing rotation of the output gear comprises meshing the first gear with the gear train.
